(19) United States
(12) Patent Application Publication
(10) Pub. No.: US 2013/0041288 A1
(43) Pub. Date: Feb. 14, 2013

(54) Apparatus and Method of Monitoring Healing and/or Assessing Mechanical Stiffness of a Bone Fracture Site or the Like

(71) Applicants: John Charles Taylor, Memphis, TN (US); Harold S. Taylor, Memphis, TN (US)

(72) Inventors: John Charles Taylor, Memphis, TN (US); Harold S. Taylor, Memphis, TN (US)

(21) Appl. No.: 13/204,793

(22) Filed: Aug. 8, 2011

(51) Int. Cl. A61B 5/103

(52) U.S. Cl. 600/587

(57) ABSTRACT

An apparatus and method of monitoring healing and/or assessing mechanical stiffness of a bone fracture or the like is disclosed. The method comprises (a) recording a strut length of a variable length strut with an indicator pin, wherein the strut is on an external fixator is in a no or minimal load situation; (b) inputting such strut length, mounting parameters, and frame parameters into software to determine a first pose and position of the fixator; (c) positioning at least one indicator clip on each side of the indicator pin; (d) applying a load or torque to the bone fracture; (e) recording strut length of the variable length strut in loaded situation; (f) inputting such strut length, mounting parameters, and frame parameters into software to determine for a second pose and position of the fixator in a loaded situation; (g) solving for a change in pose and position by subtracting the second pose and position from the first pose and position; and (h) solving for the stiffness of the bone fracture by dividing the load applied to the bone fracture by the change in position and pose. An apparatus and indicator clip are also disclosed.

### FIELD

The present disclosure relates to a method of monitoring healing and assessing mechanical stiffness of a bone fracture site or the like. The method includes using an external fixator having at least one variable length strut. The present disclosure also relates to an indicator clip used to indicate the movement of a variable length strut.

### BACKGROUND

External fixators or orthopedic fixation devices such as the device disclosed in U.S. Pat. No. 5,702,389, fully incorporated herein, are medical devices used to treat a wide variety of bone and soft tissue problems. For example, external fixators are used to stabilize bones after fracture or after osteotomy. External fixators are also used for treatment of nonunions and for other orthopedic reconstructive procedures such as limb lengthening and limb reconstruction. External fixators operate by connecting pins and/or wires to a person's bones and then connecting the pins and wires to an outer scaffolding or frame outside the affected limb. Several types of external fixators exist including but not limited to unilateral fixators, circular fixators, spatial fixators and hybrid fixators. Any configuration of external fixator may be used as desired by one skilled in the art.

Determining when a fracture site, osteotomy site, docking site, or distraction regenerate bone is healed and thereby determining when to remove the external fixator is difficult with external fixation. Healing times for bones vary greatly depending on the age and medical condition of the patient, as well as the location of the fracture, osteotomy, or nonunion and the condition of the tissues. After the external fixator is applied to a patient's affected limb, the patient is usually re-examined at one to two week intervals until the bone has been adjusted to its preferred final position, referred to as the adjustment phase. A patient is re-examined at one to two week intervals especially if the external fixator is being adjusted to realign bone fragments or lengthen the bone through distraction regeneration. Such re-examinations typically include radiographs of the affected limb in the external fixator. After the bones have been adjusted to their preferred final position, examination intervals may be increased to two to four week intervals, known as the consolidation phase. After the bone fracture or the like has healed, the external fixator is removed, sometimes in an office setting, other times in surgery under sedation or anesthesia.

The combined adjustment phase plus the consolidation phase may be up to two years or more for cases involving nonunions with missing segments of bone from open trauma, infection, tumor resection and other orthopedic reconstructive situations. Fractures in adults typically take four and a half to six months to heal in external fixation. Sometimes an external fixator may be used without additional correction other than that achieved at surgery in its initial application. However, interpretation of radiographs can be difficult and occasionally external fixators have been removed prematurely leading to additional time in a cast in the best situation or requiring reoperation with reapplication of an external fixator, or fixation with other means such as an intramedullary nail or plate in a worse situation. The tendency to leave the external fixator on a patient for extra time is tempered by the complications seen with prolonged treatments in external fixators including; pin or wire breakage, frame breakage, pin and wire site infection, limb edema, and interruption or modification of many of the activities of daily living. Some of these complications may necessitate further additional surgical procedures with attendant risks.

In addition to radiographs being difficult to interpret, radiographic examinations utilize ionizing radiation which is costly to the patient and to society as well as has a cumulative risk of ionizing radiation to the patient and medical personnel.

In addition to using radiographic appearance of a fracture site or osteotomy site or nonunion site to determine whether a fracture has healed, a physician may perform mechanical testing to determine whether a fracture has healed. Typically mechanical testing consists of a stress test or a trial period with less or no frame support. The stress test includes loosening or temporarily removing spanning components of the external fixator, and then the physician attempts to bend the bone in one direction as a radiograph is taken and then bend in the opposite direction as a second radiograph is taken. These radiographs (or transparencies) are superimposed and/or measured to see if there is relative bending of the bone displayed between the two radiographs. To ensure accuracy, the plane of the radiograph must be kept the same for the two radiographs, which can prove difficult for the physician as he attempts to bend the limb in the external fixator.

The trial period mechanical test is performed by removing part or all of the spanning components of the frame and allowing the patient to walk with no additional support at the fractured site for days to weeks. Radiographs are repeated to detect any change of position of fractured fragments as a result of walking. The same problems remain in comparing radiographs over time especially the dependence on maintaining the same plane of the radiograph and the same radiographic technique.

Prior to a stress test or trial period mechanical test, the lengths of each strut are recorded. At the conclusion of the stress test or trial period mechanical test, the struts may be returned to their original position if the physician feels more time is necessary to stiffen or strengthen the bone. Or, the struts may be adjusted to a new position based on the perceived clinical needs. For example, the physician may want to better reduce a fracture or compress/distract a nonunion. If the physician feels the bone is sufficiently healed based on the stress test or trial period mechanical test, the external fixator with its pins and wires are removed.

Fractures or osteotomies that persist over an excessive time may require additional treatment including additional surgery. Treatment may consist of an increase in weight bearing status or further adjustment of the external fixator to improve fracture reduction or fracture site compression/distraction. Electrical, electromagnetic, or acoustic (ultrasound) stimulators of bone growth have been used. Recent ultrasonic bone stimulators have been designed specifically for use with circular fixators. Some recalcitrant cases may require additional surgery to replace or augment boney fixation with wires or pins or other frame modification. Still other cases may require bone grafts applied to the site to improve healing.

There are several methods or solutions that may be used to make adjustments to an external fixator. For example, in the past, the inverse kinematic method has been used to pre-adjust an external fixator to exactly match a skeletal deformity. After the frame is attached to the bone fragments, the frame is adjusted to its corrected position thereby correcting the skeletal deformity. In orthopedics, this has been called the chronic method. The inverse kinematic method has also been used in situations after a frame with all struts at the same length has been attached to fractured bones or bones with skeletal deformity. By adjusting the frame in the opposite ways of the current skeletal deformity, the skeletal deformity is corrected while the frame is deformed in the opposite ways. In orthopedics, this has been called the residual method.

More recently the forward kinematic solution has been utilized to make adjustments to an external fixator regardless of initial strut settings to further correct skeletal deformity or address perceived clinical needs such as applying further compression to an arbitrarily adjusted frame treating a non-union of the bones.

Simplistically, six current strut settings input to the forward kinematic program yield the initial six axis pose and position of one ring with respect to the other ring. The physician measures radiographs and makes clinical measurements to determine the skeletal deformity, the pose and position of one bone fragment with respect to a second bone fragment, i.e., the deformity parameters. These deformity parameters are mathematically combined to the initial pose and position of the frame to find the final or target pose and position of the frame. The inverse kinematic method can then be used to find the corresponding final strut lengths to yield the final frame pose and position and thereby correct the attached bone fragments.

When a physician wants to determine the stiffness of the healing bone, the frame may actually be in a state of preload such as compressing a nonunion. The strut lengths are recorded to be able to return to this preferred position at the conclusion of the test if necessary.

Next, with no or minimal load on the limb, such as hanging off the edge of a table or resting gently on the floor, each variable length strut is unlocked to achieve a resting length where there is no tension or compression between the strut and the rings or partial rings to which the struts are attached, and therefore minimal or no load between the bones to which the rings or partial rings are elastically coupled via skeletal half pins, full pins, and or wires. This process is referred to as neutralizing or floating a frame. The strut lengths in this “no” load situation are recorded.

Likewise, under any particular load of interest the frame may be floated or neutralized and the corresponding strut lengths recorded. Loads may be relatively uniaxial such as axial compression, axial tension, torsion, or bending, or more complex such as oblique plane bending and axial compression. Loads may be measured with a variety of devices from bathroom scales to load cells to torque wrenches. Devices measuring loads applied to the limb may also be able to record peak load and/or load verses time in a multichannel recorder also recording strut lengths during loading of a limb.

Several methods may be used to float a frame with standard variable length struts. First, with the limb in a minimal or no load situation, all struts are removed from at least one end and reinserted usually in a slightly altered length causing no change in relative position between the rings or partial rings. Alternatively, the knurled adjustment ring can be rotated in one direction and then the other until a position is reached where the strut is not in compression or tension. This neutral or floating position can be felt and seen by observing the mechanical backlash at the couplings of the strut to the ring or partial ring and the threaded nose of the strut with respect to the threaded rod of the strut and the strut body. The strut lengths in this no load situation are recorded.

It would be desirable to provide a method of monitoring healing and assessing the mechanical stiffness of a bone fracture without using radiographs. Additionally, it would be desirable to provide a method of monitoring healing and assessing the mechanical stiffness of a bone fracture allowing shortened time in the external fixators thus decreasing complications seen with prolonged time in an external fixator. Additionally, it would be desirable to provide a method of monitoring healing and assessing the mechanical stiffness of a bone fracture that will allow early identification of fractures which are in need of additional treatment thereby shortening overall treatment times.

### BRIEF SUMMARY

An indicator clip may be used with several different types of variable length struts, and may be used with and without computer algorithms. The clip may be a part of a variable telescopic strut that has a fine adjustment. An occasional problem with fine adjustment struts is that the knurled nose of the strut body may occasionally rotate when rubbed over the patient's clothes or bedding causing an inadvertent change in strut length. The indicator clip as an add-on or a permanent part of a variable length strut would demonstrate the extent of strut drift. Problematic struts could then be exchanged or locked. The same applies to a lesser extent for FastFix struts in their fine adjustment mode.

Unlike a stress test or trial period mechanical test no radiograph is necessary to monitor healing and assess mechanical stiffness. The existing frame itself may be used as a six axis strain gauge. The method includes software using an algorithm with the forward kinematic solution yielding a change in the pose and position as a result of applied load. Displacement along and rotation about each of the three cardinal axes as a result of a given load may be calculated. Alternatively, the resultant three axis translation and the resultant three axis rotation for a given load may be calculated. All of these values help assess the mechanical stiffness and biologic healing at the fracture site. The method of monitoring and assessing may identify fractures that are not progressively healing and may require different treatments. The method of monitor healing and assessing stiffness may also identify fractures that have healed sufficiently for frame removal even though radiographs are worrisome or inconclusive and thus shorten the time in the external fixator, decreasing complications seen with prolonged time in an external fixator.

In one embodiment, an indicator clip may be a feature of a new variable length strut. In another embodiment, indicator clip may be a feature on an existing variable length strut. With struts unlocked and relatively free to move, initial strut lengths are recorded corresponding to a relatively unloaded or no load situation. Next the affected limb is subjected to a known load. The indicator clip marks the strut lengths achieved under such known load. Thus two sets of six strut lengths are generated, one in the relatively unloaded situation and one in the loaded situation. Using software including mathematical algorithms and the forward kinematic and inverse kinematic solutions for a six axis parallel manipulator external fixator, such as the Taylor Spatial Frame external fixator, the total change in position and angulation of one fragment with respect to another fragment may be determined. In the case where the bone fragments are subjected to a known load, translational stiffness and bending stiffness for each of the cardinal axes may also be generated.

Even if specific loads were not known, absolute strain associated with clinically reproducible scenarios, such as one legged stance on the fractured limb, would be informative. The information gained by the use of struts with indicator clip allows the physician to assess the mechanical properties of the fracture site. This helps the physician determine when the fracture or osteotomy is sufficiently healed for frame removal. This mechanical information can also alert the physician to fractures in need of additional treatment or time to heal.

Another clinical situation occasionally occurs during gradual correction of skeletal deformities or limb lengthening with external fixation especially in children. In some cases the bone heals before the complete correction can be accomplished. This is called premature consolidation, and usually results in repeat surgery of re-cutting the bone or under-correction of the deformity if repeat surgery is not performed. In one embodiment, the disclosed method allows the physician to gather information about the mechanical stiffness at the fracture site/osteotomy site in addition to or in lieu of radiographic examinations. This additional information may alert the physician to impending premature consolidation. If alerted, the physician may be able to increase the rate of the correction to prevent premature consolidation.

In addition to information gained from a single assessment, fracture site stiffness or specific activity associated strain, multiple assessments over time could provide important information about progression, stagnation, or even regression of healing. This information would be used to confirm adequacy of current treatment or alert the surgeon to the possible need of intervention.

In one embodiment, a patient has an external fixator with variable length struts applied for a fracture (or osteotomy or nonunion). The mechanical stiffness at the fracture site is determined as follows. In the no load or minimal load situation, the frame is floated and the corresponding strut lengths are recorded. These strut lengths as well as the usual mounting parameters and frame parameters are input into the external fixator software to solve for a first pose and position of the frame and bone fragments in the minimal or no load situation. Next, with all fast fix struts unlocked, a load is applied to the bones by the patient stepping onto a load cell (scale) with the affected limb and maintaining sufficient load or with the patient seated with the foot on the floor placing weighted saddlebags over the thigh with the hip and knee joints each flexed approximately 90°. In this loaded position, the strut lengths are recorded. Alternatively, the variable length struts are relocked under load and their respective lengths noted later. These ‘loaded’ strut lengths as well as the usual mounting parameters and frame parameters are input into the external fixator software to solve for a second pose and position of the frame and bone fragments in the loaded position. The second pose and position parameters (three angulations and three translations) of the loaded frame/bone are subtracted from the first pose and position parameters of the unloaded frame/bone. The load divided by the corresponding difference in a cardinal translation or rotation yields stiffness information. These stiffness values can be followed over time to assess healing. As a fracture heals, the displacements and bending under loads should decrease to those of an intact bone. Following these measurements over time allows the physician to identify cases that show a more typical progression to healing or cases that show an abnormally slow progression or even a regression in healing. Cases displaying abnormally slow progression or regression in healing may be further treated in the appropriate manner.

Utilizing the mounting parameters the strain at the origin may be determined, however, it may be sufficient to only measure the strain from the center of one ring to the center of the second ring, especially under relatively pure axial loading (Z-axis).

Alternatively, the patient may stand on a load cell and apply a torsional load thru the fracture site noting the unloaded and loaded lengths of struts. This also will yield data about the translations and rotations between the two fragments as a result of a torsional load.

Alternatively, a load cell can be used to apply a measurable load across the fracture in transverse shear. The frame is floated before and during load and the strut lengths recorded. This will yield the resulting translations and rotations of the bone fragments as a result of transverse force and thus a transverse stiffness.

Alternatively, using a load cell to apply a known force a measurable distance from the fracture site in a bending mode may provide torsional and bending stiffness measurements of the fracture site by noting strut lengths in the no load and loaded situation and processing through the software which solves for a difference in pose and position. The applied torque divided by translational and rotational differences yield bending/torsional stiffness information.

A load or a torque or both can be applied along or about each of the cardinal axes in either a positive or a negative direction. The unloaded strut lengths and the loaded strut lengths are recorded. This data will yield stiffness values at the fracture site.

In one embodiment, the method comprises an indicator clip or apparatus that may be attached to an existing variable length strut. In another embodiment, the method comprises a clip or apparatus that may be attached to an entirely new strut. In one embodiment, the clip indicates and retains the length of travel of a variable length strut or similar strut. Ideally, a load cell or scale with the ability to retain its maximum value until reset is utilized. Otherwise, the maximum reading of the load cell is noted. In one example embodiment, in the no load situation, the indicator clips may be positioned about the indicator pin on each strut and the corresponding strut lengths are recorded. Next, a load is applied to the bones and then released. The indicator clips on the struts will mark the maximum loaded strut lengths and the load cell or scale will retain the maximum load. Again this information is used to determine fracture site stiffness. In one embodiment, the method comprises determining strut lengths in a known load situation rather than a no load situation and the corresponding strut lengths recorded. Then a second known load may be applied and the corresponding strut lengths recorded. As long as the loads are of different magnitude, fracture stiffness information can be gained.

Currently, existing software may be used to find the forward and/or inverse kinematic solutions required to use some external fixators such as the Taylor Spatial Frame. The additional algorithms required to perform the calculations for determining fracture site stiffness may be part of the existing software. In other embodiments, local computers, PDAs, smart phones or other calculators could obviously be used.

Using this method of monitoring healing and assessing mechanical stiffness of a fracture site or the like has many significant benefits. Many of the radiographic examinations with ionizing radiation can be eliminated reducing the cost to the patient and society, and also reducing the cumulative risk of ionizing radiation to the patient and medical personnel. The method of monitoring healing and assessing the stiffness of a bone may be performed virtually anywhere, including the patient's home, decreasing the need for outpatient office follow-up. Radiographic healing often lags mechanical healing resulting in prolonged treatment times in external fixators. With the method of monitoring healing and assessing mechanical stiffness, treatment times may decrease if the physician feels the fracture site has achieved certain mechanical milestones even though radiographic healing is still incomplete.

Importantly, fractures at risk may be identified earlier by a failure to stiffen or even a decrease in stiffness at the fracture site over time. This could lead to earlier intervention and overall decreased time in the external fixator versus those followed conventionally with a delayed response for additional treatment.

In one embodiment, the method involves attaching an external fixator (such as a Taylor Spatial Frame) or similar device mechanical sensor to another (second) external fixator (such as a unilateral fixator) which is attached to the skeleton. The spanning members of the second external fixator are removed or thoroughly loosened. The offsets of the accessory or piggyback external fixator are recorded under mounting parameters and the method may be used to determine two or more sets of strut lengths under two or more different loads. Again, each set of strut lengths yields a pose and position via calculations. The loaded pose and positions are subtracted from the unloaded pose and position to find a difference or change in each of the pose and position components as a result of the load. This strain information combined with the difference in loads yields a measure of fracture site stiffness.

In yet a further embodiment, the method involves using an electric/electronic strut, for instance with a linear variable differential transformer (LVDT), to electronically determine strut length and retain the limit of mechanical travel or to transmit this data to an electronic recorder or computer. Other electronic, optical, electro/optical means are available to measure position/travel. This data when combined with the applied load will yield important fracture stiffness information. Strut lengths and applied loads to a load cell could also be monitored with a multichannel recorder and even directly fed into the algorithm to determine stiffness.

Also, telescopic unilateral external fixators, such as a Hexfix, can be floated in a no load and loaded situation and a change in length recorded by an indicator clip. This change in length under load could at least yield axial stiffness (usually Z axis).

### BRIEF DESCRIPTION OF THE DRAWINGS

The present disclosure will be better understood by reference to the following detailed description when considered in conjunction with the accompanying drawings wherein:

FIG. 1 depicts three perspective views of an external fixator applied to a leg using skeletal pins. The bone fragments are not anatomically positioned.

FIG. 2 depicts three perspective views of an external fixator applied to a leg using skeletal pins. The bone fragments are anatomically positioned.

FIG. 3 is a perspective view of a six axes parallel manipulator type external fixator applied to the skeleton by pins and wires, wherein pins and wires are not shown.

FIG. 3A is a perspective view of a six axes parallel manipulator type external fixator applied to the skeleton by pins and wires, wherein pins and wires are not shown. A weight is applied at the ankle.

FIG. 3B is a close up of 3A.

FIG. 4 is a close up perspective view of a variable length strut in the locked position.

FIG. 5 is a close up perspective view of a variable length strut in the unlocked position.

FIG. 6 is a compilation of perspective views of an external fixator having two rings and six variable length struts.

FIG. 7 is a perspective view of two variable length struts.

FIG. 8 is three perspective views of a six axes parallel manipulator type external fixators, wherein the struts are in the unlocked position.

FIG. 9A is a side perspective view of an indicator clip according to one example embodiment.

FIG. 9B is a top perspective view of the indicator clip shown in FIG. 9A.

FIG. 9C is a perspective view of the indicator clip shown in FIG. 9A.

FIG. 9D is a perspective view of a variable length strut with indicator clips attached elastically about the strut.

FIG. 9E is a perspective view of an indicator clip according to a second example embodiment.

FIG. 10 is a perspective view of an external fixator having two rings and six variable length struts with two indicator clips elastically attached to either sides of the indicator pin.

FIG. 11 illustrates the chronic method of skeletal deformity correction.

FIG. 12 illustrates the residual method of skeletal deformity correction.

FIG. 13 illustrates the total residual method of skeletal deformity correction.

FIG. 14 is a flow chart of existing mathematical algorithms or computer programs used to control the external fixators for subsequent fracture reduction or correction of skeletal deformity.

FIG. 15A is a perspective drawing of the indicator clip for a bar or hybrid fixator.

FIG. 15B is a perspective drawing of the HEX-FIX external fixator treating a tibial fracture with indicator clips applied. The bone is not loaded axially.

FIG. 15C illustrates an applied axial load to the bone fixed with the external fixator thereby compressing the fracture site.

FIG. 15D illustrates the axial load released from the bone fixed by an external fixator. The elastic fracture site has returned to its unloaded position. The indicator clip remains in the displaced position.

FIG. 15E, F, G are close up perspectives of FIG. 15 B, C, D respectively.

### DETAILED DESCRIPTION

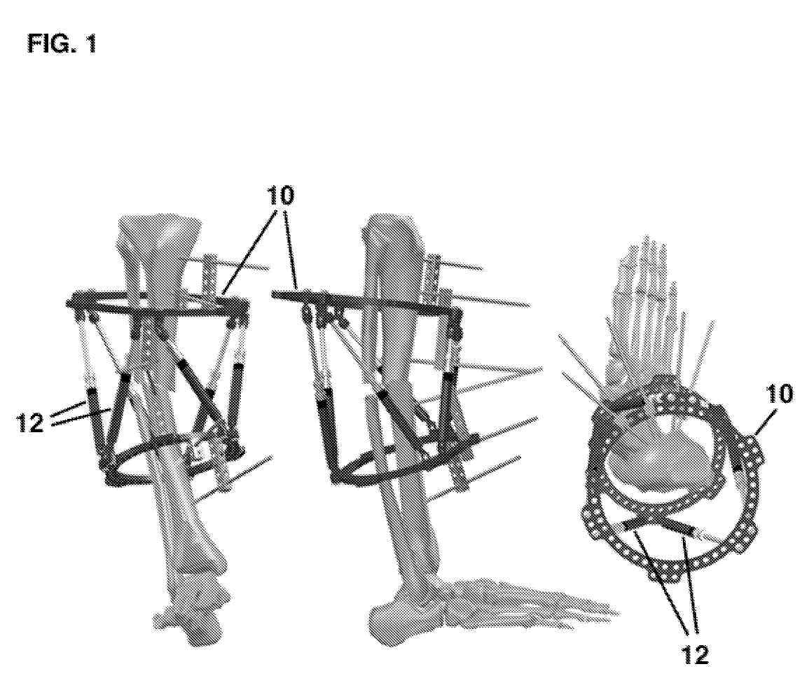

Referring to FIGS. 1 to 8, the apparatus and method of monitoring healing and/or assessing mechanical stiffness of a bone fracture site or the like comprise an external fixator 10 applied or attached to a bone fracture. External fixator 10 has at least one variable length struts 12 and may be attached to a bone fracture or the like with pins and/or wires. In one embodiment, external fixator 10 is a six axis parallel manipulator type external fixator having a plurality of variable length struts. Any type of external fixator 10, such as unilateral, circular, spatial or hybrid external fixator, may be used as desired by one skilled in the art.

In one embodiment, a unilateral fixator may consist of one bar or tube with pin clamps. In one embodiment, a unilateral fixator may have pin clamps intermediate to the ends of the bar. In another embodiment, a unilateral fixator may have pin clamps at the ends of the bar. In one embodiment, a unilateral fixator may be used for lengthening a bone. In one embodiment, a unilateral fixator may be coupled in some systems with bar to bar connections, which may be referred to a multilateral fixators.

In one embodiment, a circular fixator may consist of rings or partial rings connected by various threaded rods and/or struts. Standard attachments for circular fixators may include hinges and translation devices which allow correction of more complex angular deformity. In one embodiment, a hybrid fixator may consist of attaching rings to bars in a given construct. The rings may be useful to allow wire fixation for smaller fragments. The bars may be useful to allow fixation that is less cumbersome than circumferential rings. In another embodiment, a spatial external fixator may be a circular external fixator which may be a part of a hybrid frame, such as the Taylor Spatial Frame. The spatial frame may be capable of correcting the most complex deformity simultaneously or in steps without needing separate or serial rotation and translation mechanisms.

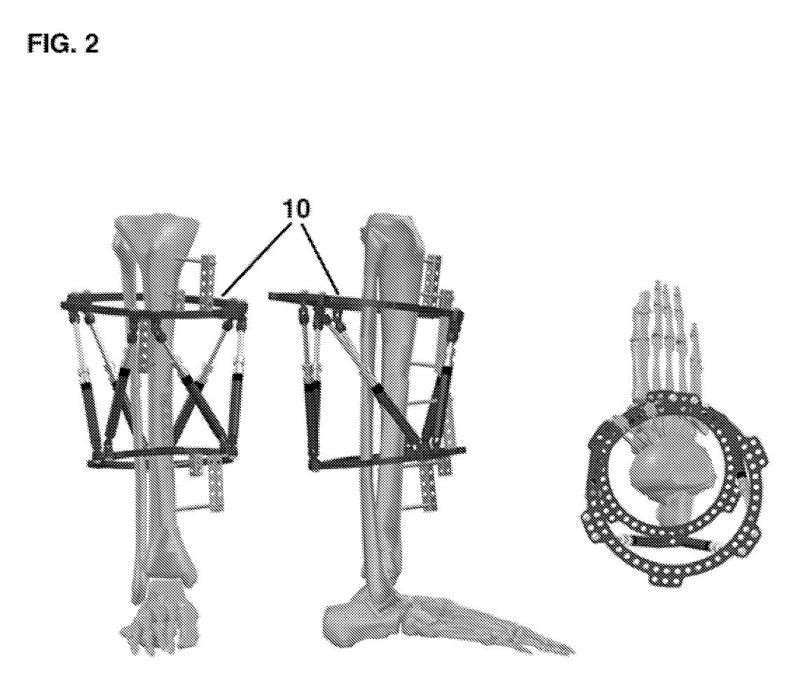

Referring to FIG. 1, in one embodiment, an external fixator 10 is applied to a leg using skeletal pins. The bone fragments are not anatomically positioned. FIG. 1 illustrates the AP or frontal view, the lateral or side view, and the axial view. Referring to FIG. 2, in another embodiment, an external fixator 10 is applied to a leg using skeletal pins. The bone fragments have been anatomically positioned. FIG. 2 illustrates the frontal view, the lateral or side view, and the axial view.

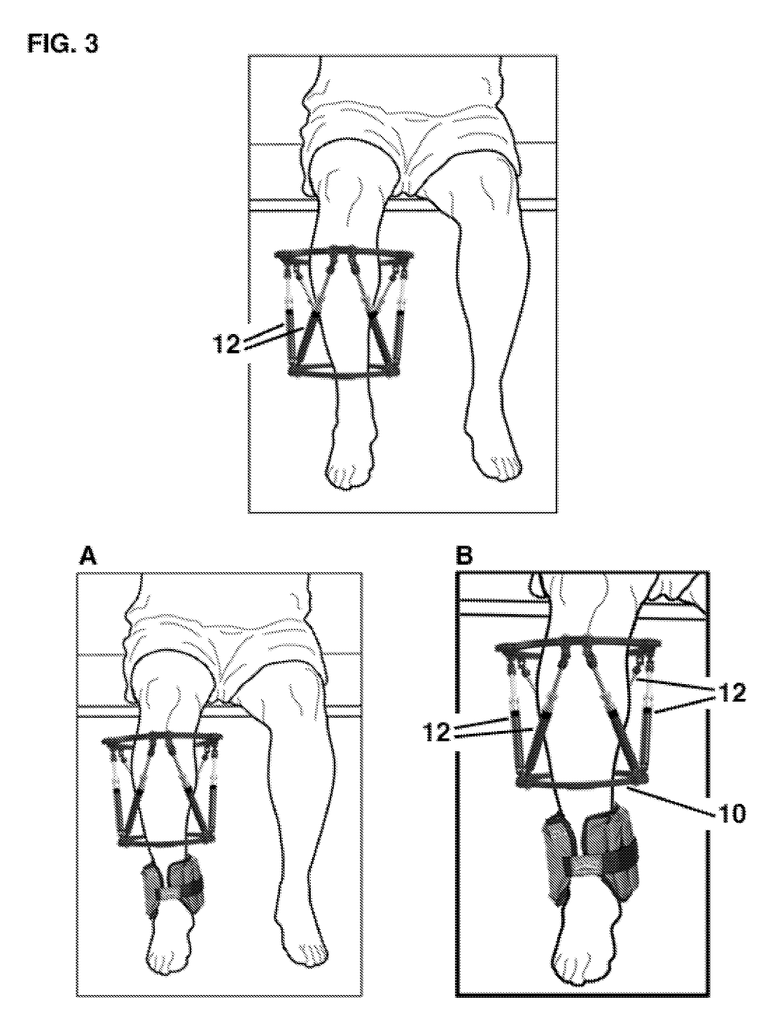

Referring to FIG. 3, in one exemplary embodiment, a six axes parallel manipulator type external fixator 10 may be applied to the bone fracture or the like by pins and or wires. (Pins and wires omitted for clarity.) FIG. 3 illustrates a relatively unloaded limb in an external fixator hanging off a seat or table. FIGS. 3A and 3B show an additional weight applied to the limb distal to the fracture which would apply a tensile force to the limb.

Any configurations of external fixator may be used as desired by one skilled in the art. Such configurations may include extra rings and/or attached unilateral external fixation components, attachments, and enhancements aiding in the treatment of skeletal and soft tissue problems including soft tissue and/or bone defects, deformities, nonunions, fractures, and short stature. Multiple problems may exist in a single boney segment requiring multiple sites of treatments, for example a skeletal nonunion with shortening may be treated at two sites; one site for the treatment of nonunion and a remote site with osteotomy and distraction osteogenesis. Progressive healing of the nonunion and healing of the osteotomy site could be monitored with the method and devices of these patents.

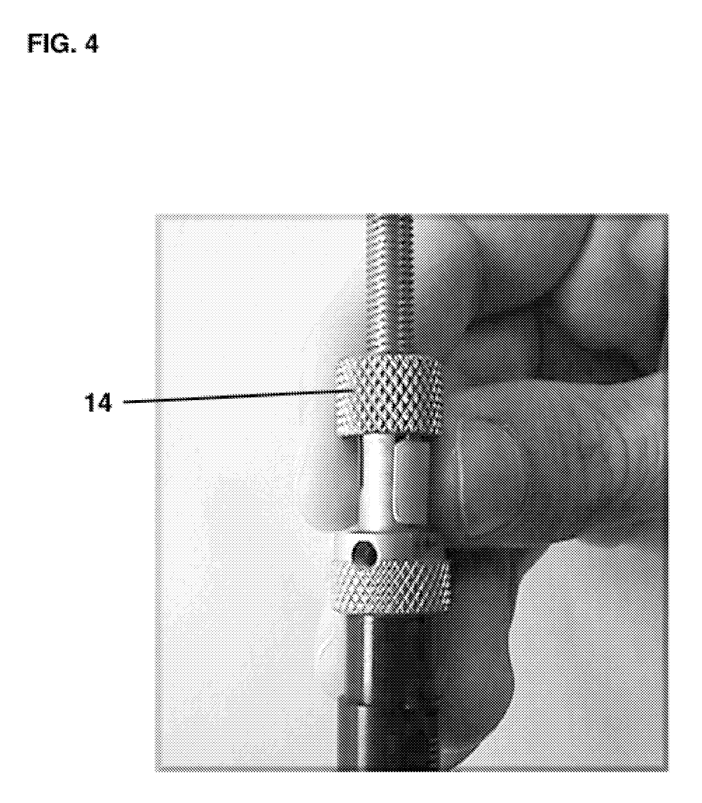

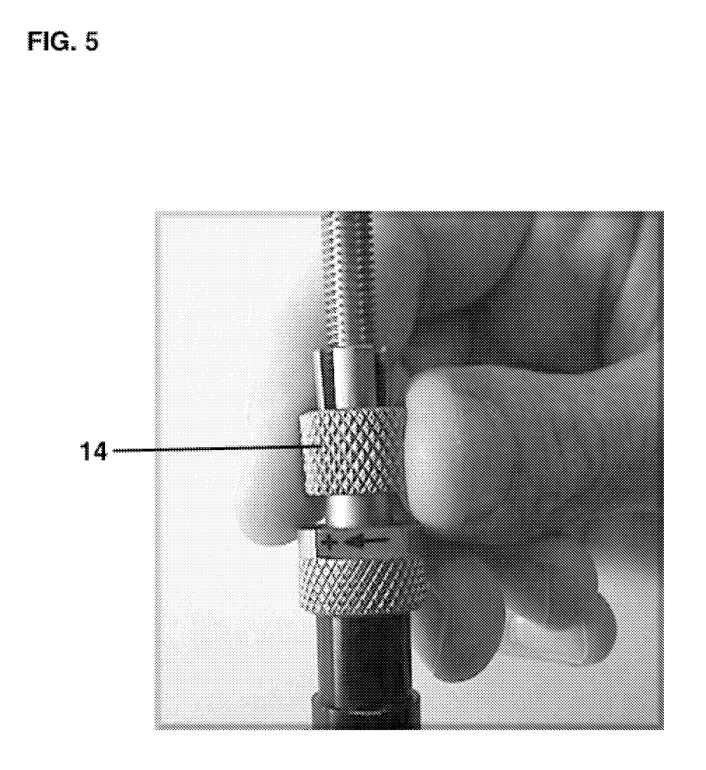

In one embodiment, a variable length strut 12 may be unlocked or locked. Referring now to FIG. 4, a close-up view of a variable length strut 12 in the locked position is shown. In this locked position no significant change in length of the strut will occur with axial loading, either compressive or distractive. FIG. 5 shows a close-up view of a variable length strut 12 in the unlocked position. The knurled retaining ring 14 has been moved to disengage female threads from the male threaded rod. In the unlocked position, the length of strut 12 will change with axial loading, either compressive or distractive. Any other telescopic struts or sliding pairs which allow a change of length with axial loading may be used as desired by one of skill in the art.

Referring again to FIG. 3, in one embodiment, six variable length struts 12 are in the unlocked position, the bone fracture or osteotomy site is experiencing a small tensile force due to the weight of the distal portion of the limb modified by muscular contractions. This reflects a no load or minimal situation or condition of the bone fracture or the like. Other rest positions are useful such as the limb being supported by a horizontal surface.

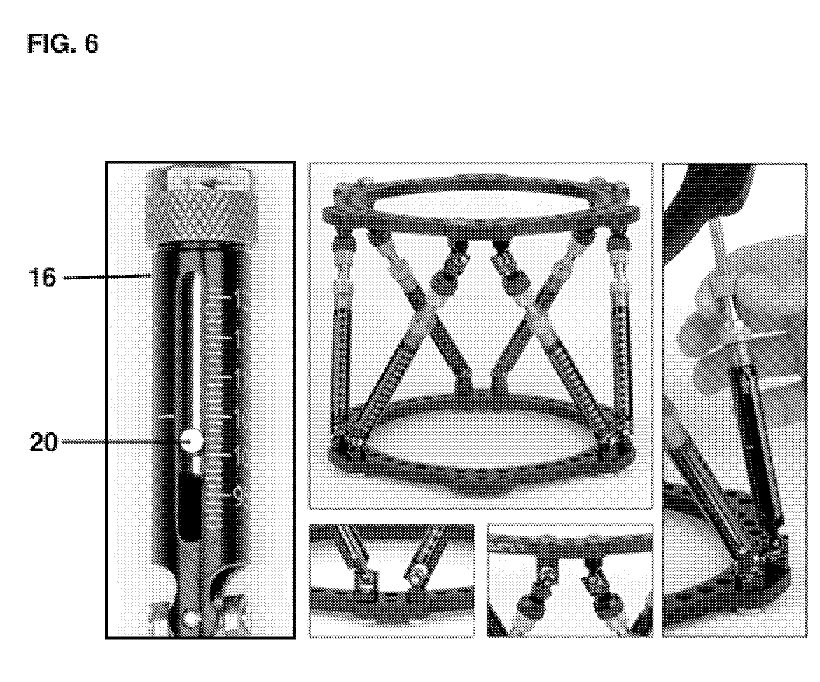

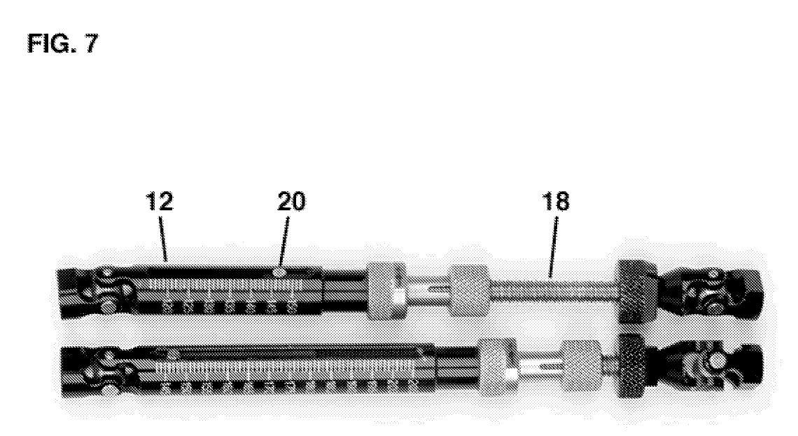

Referring now to FIGS. 6 and 7, in an example embodiment, external fixator 10 may comprise six variable length struts 12 which are used to connect two rings or partial rings. The rings are connected to the skeletal fragments by pins, wires, and clamps. By adjusting the lengths of the six struts 12 the skeletal or bone fragments may be moved as needed in all six axes (three translations and three rotations). In one example embodiment, a shoulder bolt may be used to connect each end of a strut 12 to a ring. At each end of the strut is a U-joint. At one end of the strut is the cylindrical body with indicia that gives the length of the strut 12. In one example embodiment, at the other end of strut 12 is a threaded rod portion 18 which telescopes into the cylindrical body 16. An indicator pin 20 is attached to the threaded rod 18. The length of the strut 12 is indicated by the position of the scribed line on the end of the indicator pin 20 along the numbered indicia on the strut body 16. The indicator pin 20 travels along a channel cut in the body 16 of the strut 12. In the locked position, the length of the strut can be changed gradually by turning the nose of the body of the strut, which acts as a screw jack with the threaded rod 18 portion of the strut 12.

Referring to again to FIG. 7, two variable length struts 12 are shown. Each extreme end of the variable length strut is tapped to accept shoulder bolts which are used to attach struts to rings and/or partial rings. Just inboard from each tapped end of the strut are universal joints. Just inboard from the universal joints at one end is the strut body 16 which is hollow to accept a threaded portion 18 of the strut 12. The strut body 16 may have a longitudinal slot to accept a pin 20 which prevents rotation of the threaded portion within the body and also acts as a length indicator pin. Just inboard from the universal joint at the other end is a threaded portion 18 of strut 12. This threaded portion of the strut is partially telescopically contained within the body of the strut. At the opposite end of the strut body from its universal joints is a portion of the strut body which is free to rotate about the threaded rod thereby changing the length of the threaded rod accordingly. Furthermore, this threaded portion of the body has a locking ring which may be released thereby disengaging the threaded portion of the body from the threaded rod and allowing the threaded rod to freely translate within the body of the strut. Along the length of the slot which is cut to accept the indicator pin, the strut body is marked in millimeters and centimeters and half centimeters with indicia. These markings give the current length of the strut from the center of one universal joint to the center of the opposite universal joint.

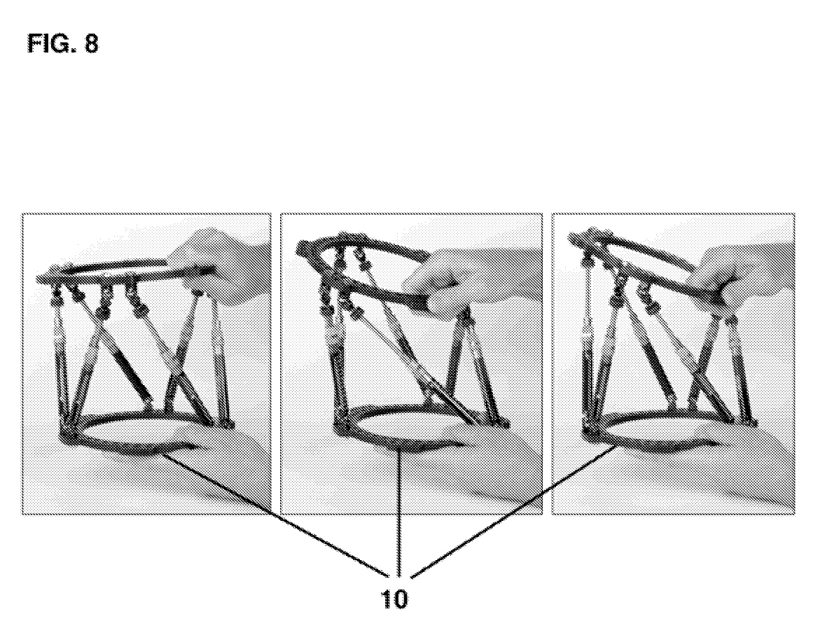

Referring now to FIG. 8, in the unlocked position, the length of the struts can be modified by applying a compressive or tensile load to the struts.

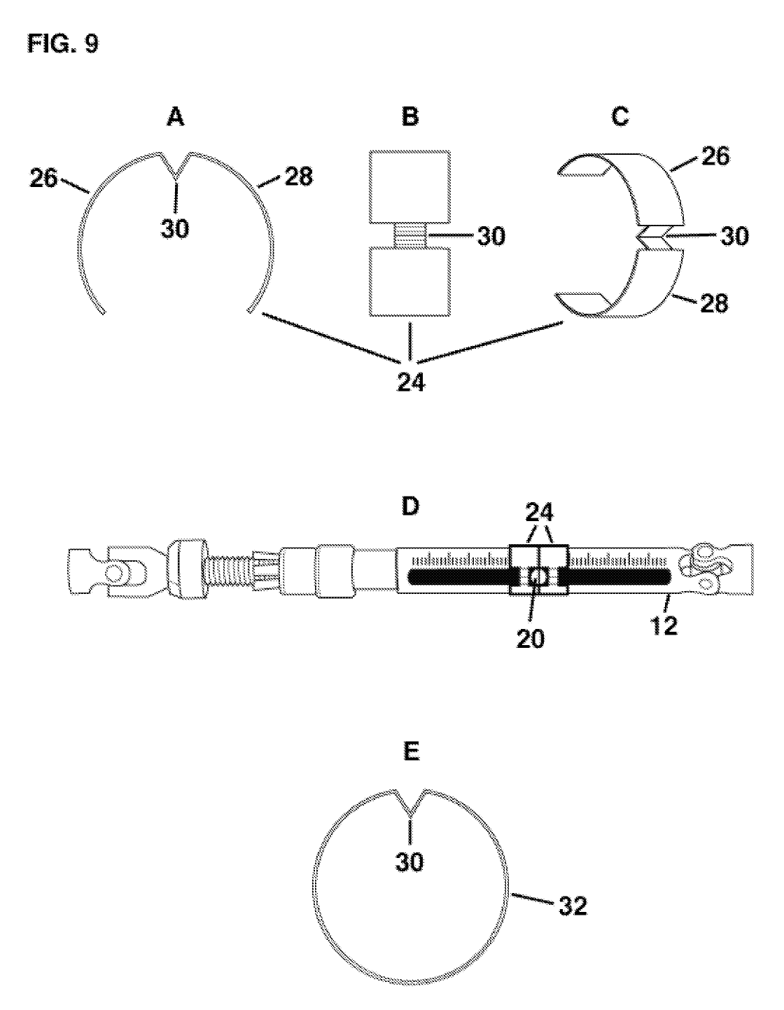

Referring now to FIGS. 9A, 9B and 9C, indicator clip 24 may be used with several different types of variable length struts, and may be used with and without computer algorithms. The clip may be a part of a variable telescopic strut that has a fine adjustment. An occasional problem with fine adjustment struts is that the knurled nose of the strut body may occasionally rotate when rubbed over the patient's clothes or bedding causing an inadvertent change in strut length. The indicator clip as an add-on or a permanent part of a variable length strut would demonstrate the extent of strut drift. Problematic struts could then be exchanged or locked. The same applies to a lesser extent for FastFix struts in their fine adjustment mode.

In one embodiment, an indicator clip used to indicate the change in length of a variable length strut comprises a first curved portion 26, second curved portion 28, and a tab 30. In one embodiment, first curved portion 24 and second curved portion 28 are configured to abut the body of the strut. In one embodiment, clip 24 which attaches elastically about the body of the variable length strut. Gentle pressure against the clip 24 along the longitudinal axis of the strut will slide the clip. Stiction friction or mechanical interference prevents the clip 24 from sliding with gravity or insignificant centrifugal force. In one embodiment, the clip 24 comprises a tab 30 contiguous to first and second curved portions 26 and 28. In an example embodiment, tab 30 is configured to reside and travel in the channel cut in the body of the variable length strut. The tab 30 of the clip 24 abuts the indicator pin attached to the threaded portion of the telescopic strut. With the tab 30 abutting the indicator pin, the edge of the clip 30 is at the same indicial mark as the length indicator pin. In one embodiment, the tab of the clip may be set back from the edge of the clip possibly one half the diameter of the indicator pin (shown in FIG. 9B). In one embodiment, in order to record the change in strut length under a loaded situation, two clips 24 may be used one on either side of the indicator pin. The clips 24 may indicate either shortening or lengthening of the telescopic strut. In one embodiment, clip 24 is permanently attached to the strut. In another embodiment, clip 24 is removably attached to the strut.

Referring now to FIG. 9E, in yet another embodiment, clip 24 comprises a first and second curved portion that make a cylindrical portion 32. In such embodiment, the clip 24 would not have an opening. Cylindrical portion 32 is configured to abut the body of the strut when attached to the strut. Tab 30 is contiguous to cylindrical portion 32. In one embodiment, clip 24 is permanently attached to the strut. In another embodiment, clip 24 is removably attached to the strut.

Referring now to FIGS. 9A, 9B, 9C and 9D, three views (axial, side, and oblique) of an indicator clip and a variable length strut with two indicator clips are applied, one on either side of the strut length indicator pin. In one embodiment, the clip is made of metal, but any other material may be used as desired by one skilled in the art. The clips may be formed in such a way that they could be applied to the strut from the end or side. In one embodiment, the clip may be springy and is applied to the side. There is sufficient stiction friction of the clip to the barrel to prevent inadvertent translation of the clip, unless pushed by the indicator pin.

In one embodiment, the indicator clip comprises a tab 30 configured to project into the slot of the strut body. In another embodiment, clip 24 30 may not have tab 30 if the indicator pin projects sufficiently out of the body slot. In one embodiment, tab 30 of the indicator clip is adjacent to the indicator pin and is notched half the diameter of the indicator pin, so that the interior edge of the indicator clip can be read against the indicia to obtain a strut length as a result of load. Other configurations of clip 24 and tab 30 with different offsets between the edge of the indicator clip and the indicator pin and even additional indicia specific for the indicator clip may be used as desired by one skilled in the art.

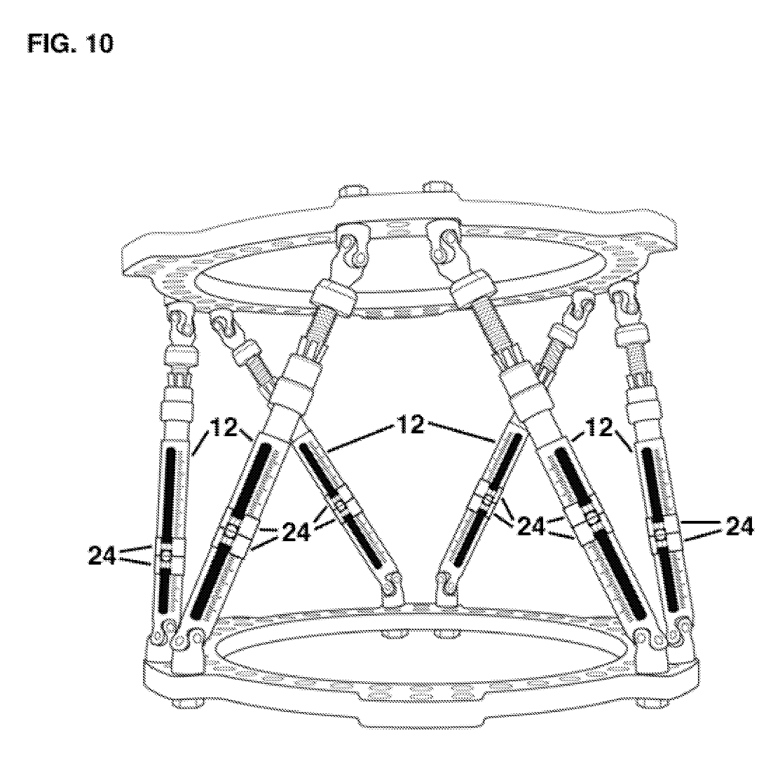

Referring now to FIG. 10, an apparatus for monitoring healing and/or assessing mechanical stiffness of a bone fracture or the like is disclosed. In one embodiment, the apparatus comprises a variable length strut and at least one indicator clip 24. The variable length strut may be attached to an external fixator 10. Strut comprises a strut body which is hollow to accept a threaded portion which telescopes into the strut body. An indicator pin 20 is attached to the threaded portion and travels along a channel cut in the body of the strut. Indicator clip 24 is attached to at least one side configured to abut to at least one side of the indicator pin of the strut. The clip comprises a first curved portion, second curved portion and a tab contiguous to the curved portions. The curved portions are configured to abut the body of the strut when attached to the strut. The tab may be configured to reside and travel in the channel located in the body of the strut when the tab is attached to the strut.

In one embodiment, the external fixator comprises a six axis parallel manipulator type external fixator having a plurality of variable length struts. In another embodiment, the external fixator may be attached to a second external fixator which is attached to bone surrounding the bone fracture. In one embodiment, the method involves attaching an external fixator (such as a Taylor Spatial Frame) or similar device mechanical sensor to another (second) external fixator (such as a unilateral fixator) which is attached to the skeleton. The spanning members of the second external fixator are removed or thoroughly loosened. The offsets of the accessory or piggyback external fixator are recorded under mounting parameters and the method may be used to determine two or more sets of strut lengths under two or more different loads. Again, each set of strut lengths yields a pose and position via calculations. The loaded pose and positions are subtracted from the unloaded pose and position to find a difference or change in each of the pose and position components as a result of the load. This strain information combined with the difference in loads yields a measure of fracture site stiffness. In one embodiment, the second external fixator may be unilateral, circular, spatial or hybrid external fixator. In one embodiment, clip may be permanently attached to the strut. In another embodiment, clip may be removably attached to the strut. In one embodiment, one indicator clip is attached to one side of the indicator pin. In another embodiment, one indicator clip is attached to each side of the indicator pin.

Referring again to FIG. 10, in one embodiment, six variable length struts may be attached to two rings or partial rings or even plates as described in the original patent. Adjustment of the six variable length struts changes the pose and position of one ring with respect to the other. The pose of an object may be the three dimensional angular orientation of the object in a frame of reference, i.e. the varus/valgus, flexion/extension and internal rotation/external rotation. The position of an object is the three dimensional coordinates of the object in a frame of reference, i.e. x, y, and z coordinates of some point of interest, usually the corresponding point.

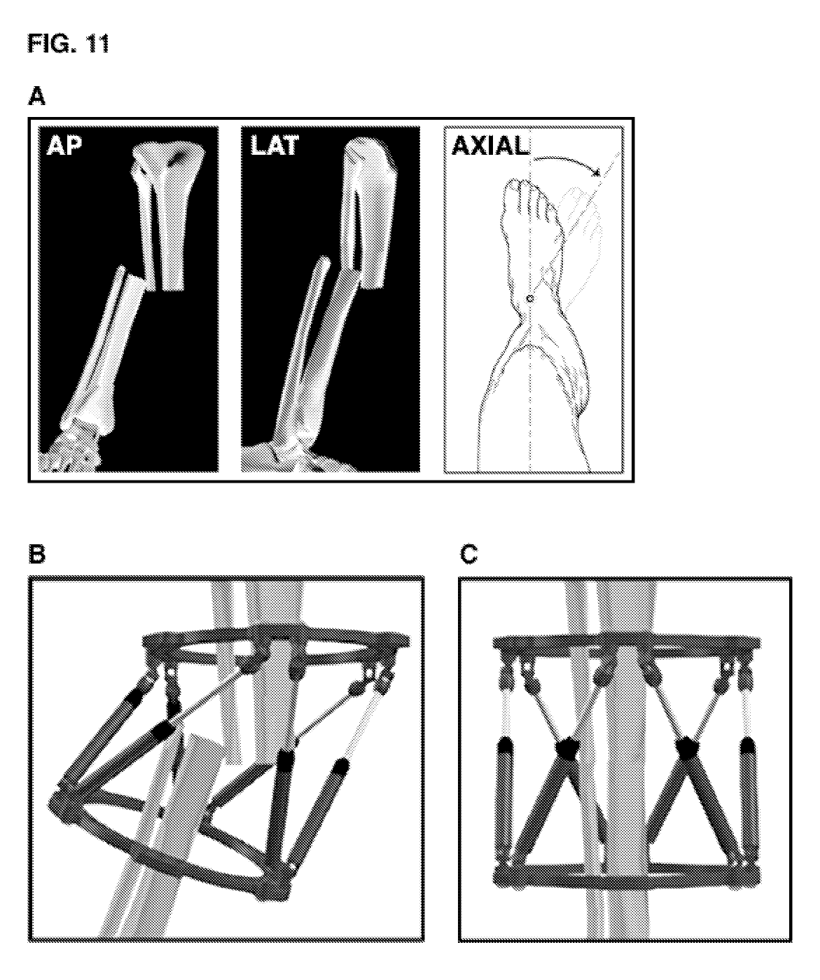

Several methods may be used to correct a skeletal deformity. Referring now to FIG. 11, the chronic method of skeletal deformity correction is illustrated. For deformity correction, the surgeon measures AP and lateral radiographs and performs a clinical exam which yield the six deformity parameters. Clinical exam determines which ring diameters to use and whether longer or shorter struts are required—the three frame parameters. The surgeon anticipates the position of the frame with respect to the origin usually the interior end of the reference fragment, thus providing the four mounting parameters. These thirteen parameters are input to a Chronic Deformity Correction Program which returns six specific strut lengths to adjust the external fixator to exactly mimic the deformity. The frame is then attached to the skeleton. The deformity will be fully corrected when the struts are restored to their neutral length. This technique is covered more fully in U.S. Pat. No. 5,702,389, incorporated herein by reference.

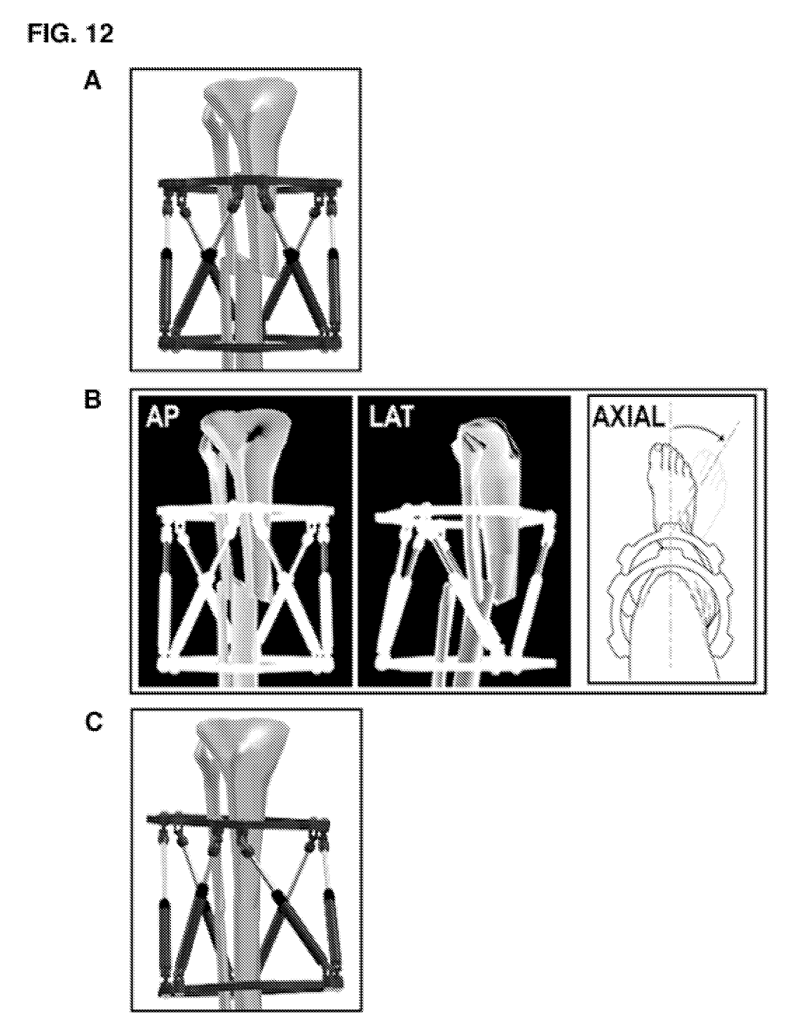

Referring now to FIG. 12, the residual method of skeletal deformity correction is illustrated. For standard acute fracture treatment, the surgeon applies an appropriately sized frame with the struts at neutral length frame parameters. Utilize standard reduction techniques as the neutral frame is applied. Postoperatively, AP and lateral radiographs are obtained and a clinical exam is performed (see B in FIG. 12). From these radiographs the six fracture deformity parameters and the four mounting parameters are measured. These thirteen parameters are input to a Residual Deformity Correction Program which returns six specific strut lengths to adjust the Spatial Frame to exactly mirror the deformity. The deformity will be fully corrected when the struts are moved to their specified lengths. Residual deformity correction may be used to further improve a chronic deformity correction or the rings first method.

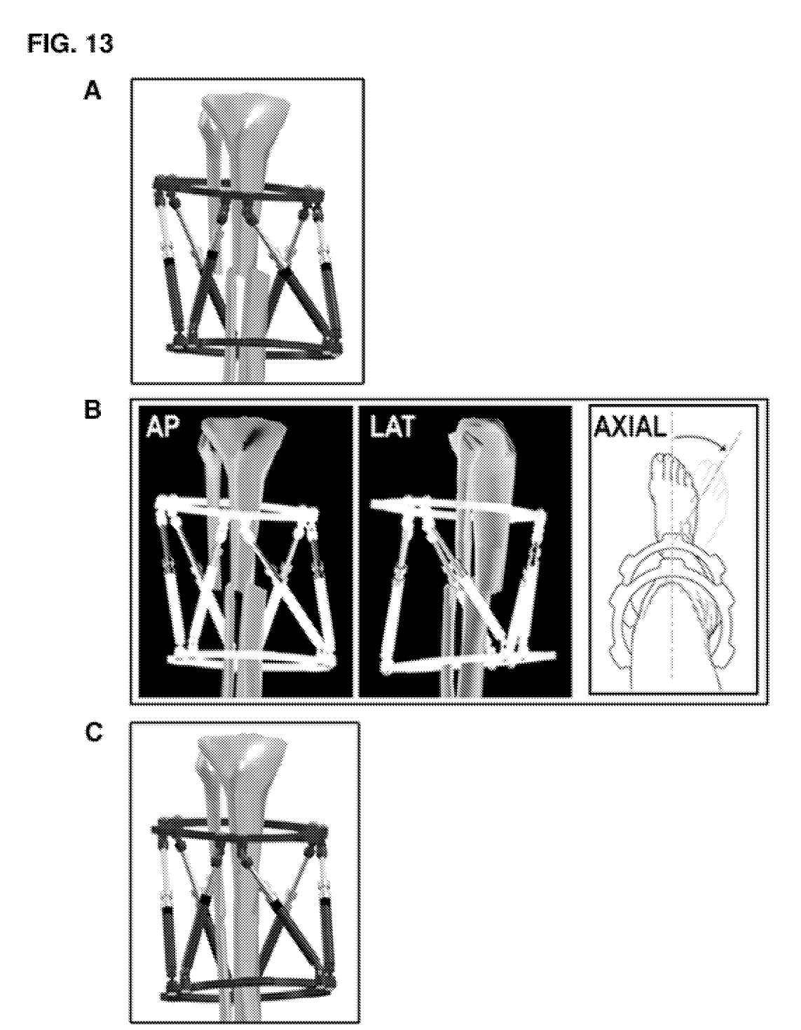

Referring now to FIG. 13, the total residual method of skeletal deformity correction is illustrated. The total residual method utilizes computer programs that perform the forward kinematic solution for a parallel manipulator. The surgeon selects one bony fragment as reference and the other fragment as the deformed fragment. The surgeon selects an origin and a corresponding point. From radiographic and clinical exam the deformity parameters are measured and input. The position of the reference ring or partial ring is measured radiographically and clinically with respect to the origin. These mounting parameters are input. The current strut lengths are measured and input. Using the forward kinematic program, the six strut lengths and ring sizes may be used to determine a pose and position of one ring with respect to the other. The pose and position of one ring with respect to the other is combined with feet deformity parameters to yield a combined pose and position as results of initial frame adjustments and persistent deformity parameters of the skeleton. This combined pose and position are usually fed into the inverse kinematic program to solve for a new set of six strut lengths that will correct the current skeletal deformity.

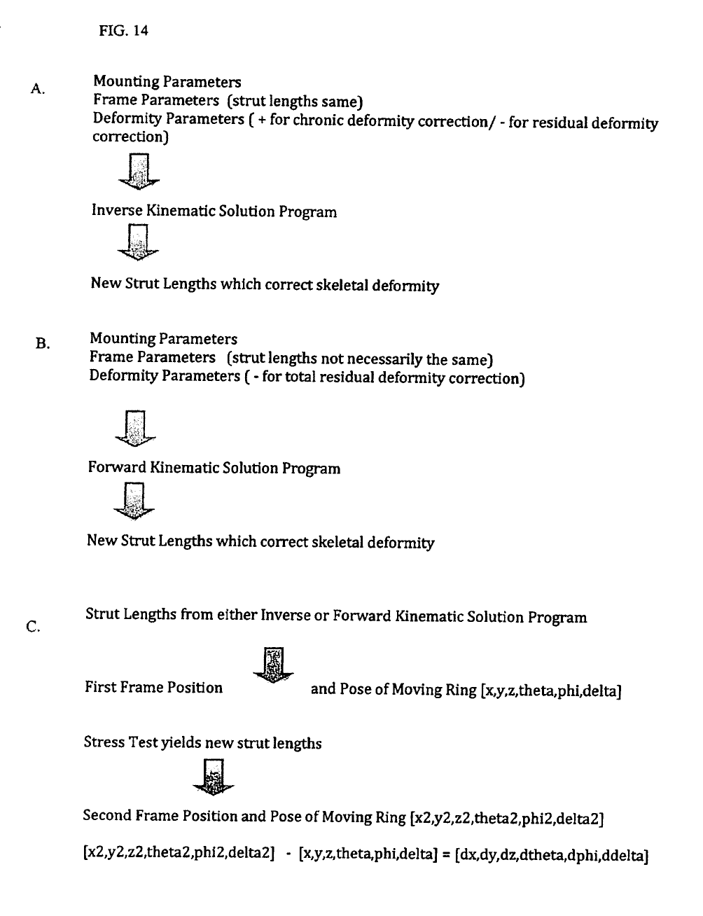

Referring now to FIG. 14, a flow chart of mathematical algorithms or computer programs used to control an external fixator for subsequent fracture reduction or correction of skeletal deformity is disclosed. In the past, mathematical algorithms or computer programs have only been used to provide strut lengths to either mimic a skeletal deformity, or to correct a skeletal deformity while attached to the spatial frame external fixator.

The associated mathematical algorithm or computer program with the indicator clip transforms an external fixator into a six axis measuring device, which when combined with the associated loads that in general produce a six axis displacement, yields information about the mechanical characteristics at the fracture site. Prior mathematical algorithms and computer programs associated with spatial frame external fixators could not yield information about stiffness at the fracture or osteotomy site.

The software for controlling a spatial frame external fixator, such as SPATIALFRAME.COM VERSION 3.1, as well as other engineering programs that solve for the forward kinematic solution of a Stewart Platform, have as part of their programs a subroutine that given six strut lengths will solve for the position and pose ‘attitude’ of one ring with respect to the reference ring. This subroutine is utilized not simply to adjust struts to correct chronic or residual deformity, but to determine strain at the fracture/delayed union site in conjunction with the indicator clips.

A method of monitoring healing and/or assessing mechanical stiffness of a bone fracture or the like is disclosed. In one embodiment, the method comprises recording a strut length of at least one variable length strut with an indicator pin. The strut is on an external fixator attached to a bone surrounding a bone fracture. The external fixator is in a no load or minimal load situation or position. In order to achieve a no load or minimal load position, the limb is placed in a relatively unloaded or no load position. The at least one strut may be placed in its unlocked position.

With the limb in a relatively unloaded situation, the lengths of the six struts are recorded. The strut lengths along with the mounting parameters and frame parameters are input into software to determine for the first pose and position of the fixator in the no load or minimal load situation. These six strut lengths in the unloaded situation are input to the subroutine which yields the x, y, and z coordinates of the center of the moving ring and the attitude of the moving ring about these same axes:
First (unloaded) Position and Pose of Moving Ring=[x₁, y₁, z₁, theta₁, phi₁, delta₁].

In one embodiment, an indicator clip may be positioned on each side of the indicator pins (usually all six). In another embodiment, an indicator clip may be positioned on one side of the indicator pin (usually all six). In still another embodiment, indicator clips are not used.

With each of the struts in the unlocked position, the strut may lengthen or shorten as a result of axial load, either compressive or tensile. In one embodiment, one or the other indicator clips for each indicator pin may have been pushed into a new position by the indicator pin. These new positions are recorded and used in software to determine a change along the six cardinal axes as a result of the given load.

In one embodiment, a load may be applied to the fractured bone by the patient stepping onto a load cell (scale) with the affected limb and maintaining sufficient load or with the patient seated with the foot on the floor placing weighted saddlebags over the thigh with the hip and knee joints each flexed approximately 90°. In this loaded position, the strut lengths are recorded. Alternatively, the variable length struts are relocked under load and their respective lengths noted later. These ‘loaded’ strut lengths as well as the usual mounting parameters and frame parameters are input into the external fixator software to solve for a second pose and position of the frame and bone fragments in the loaded position. The second pose and position parameters (three angulations and three translations) of the loaded frame/bone are subtracted from the first pose and position parameters of the unloaded frame/bone.

Alternatively, the patient may stand on a load cell and apply a torsional load thru the fracture site noting the unloaded and loaded lengths of struts. This also will yield data about the translations and rotations between the two fragments as a result of a torsional load.

Alternatively, a load cell can be used to apply a measurable load across the fracture in transverse shear. The frame is floated before and during load and the strut lengths recorded. This will yield the resulting translations and rotations of the bone fragments as a result of transverse force and thus a transverse stiffness.

Alternatively, using a load cell to apply a known force a measurable distance from the fracture site in a bending mode may provide torsional and bending stiffness measurements of the fracture site by noting strut lengths in the no load and loaded situation and processing through the software which solves for a difference in pose and position. The applied torque divided by translational and rotational differences yield bending/torsional stiffness information.

A load or a torque or both can be applied along or about each of the cardinal axes in either a positive or a negative direction. The unloaded strut lengths and the loaded strut lengths are recorded. This data will yield stiffness values at the fracture site.

The appropriate load, L, is applied to the limb. In general the load results in a change in length of the six struts. The change in lengths is indicated by the positions of the indicator clips. The lengths of the six struts under load are recorded and input, along with the mounting parameters and frame parameters, to the subroutine which yields the x, y, and z coordinates of the center of the moving ring and the attitude of the moving ring about these same axes in the loaded situation:
Second (loaded) Position and Pose of Moving Ring=[x₂, y₂, z₂, theta₂, phi₂, delta₂].

The second position and pose of the moving ring is subtracted from the first position and pose to yield a change in position and pose along and about each of the cardinal axes, essentially strain along and about the axes as a result of the load applied.

[x₁, y₁, z₁, theta₁, phi₁, delta₁]−[x₂, y₂, z₂, theta₂, phi₂, delta₂]=strain

The load, L, applied divided by strain yields the stiffness at the fracture/osteotomy site. Utilizing the mounting parameters the strain at the origin may be determined, however, it may be sufficient to only measure the strain from the center of one ring to the center of the second ring, especially under relatively pure axial loading (Z-axis). The orthogonal components of the fracture stiffness can be investigated based on the design of the test. Other clinical tests may be performed to determine change of strut length as indicated, such as ambulation, arising from a chair, running, or rowing.

Another method involves using an electric/ electronic strut, for instance with a linear variable differential transformer (LVDT), to electronically determine strut length and retain the limit of mechanical travel or to transmit this data to an electronic recorder or computer. Other electronic, optical, electro/optical means are available to measure position/travel. This data when combined with the applied load will yield important fracture stiffness information. Strut lengths and applied loads to a load cell could also be continuously or frequently monitored with a multichannel recorder and even directly fed into the software to determine stiffness.

In one embodiment, the method comprises the initial step of recording initial strut length of at least one variable length strut. In another embodiment, the steps of the method are repeated as needed to monitor healing and/or assess mechanical stiffness of a bone fracture. These stiffness values can be followed over time to assess healing. As a fracture heals, the displacements and bending under loads should decrease to those of an intact bone. Following these measurements over time allows the physician to identify cases that show a more typical progression to healing or cases that show an abnormally slow progression or even a regression in healing. Cases displaying abnormally slow progression or regression in healing may be further treated in the appropriate manner.

In still another embodiment, the external fixator comprises a six axis parallel manipulator type external fixator having a plurality of variable length struts. In a further embodiment, the external fixator is attached to a second external fixator which is attached to bone surrounding the bone fracture. In one embodiment, the method involves attaching an external fixator (such as a Taylor Spatial Frame) or similar device mechanical sensor to another (second) external fixator (such as a unilateral fixator) which is attached to the skeleton. The spanning members of the second external fixator are removed or thoroughly loosened. The offsets of the accessory or piggyback external fixator are recorded under mounting parameters and the method may be used to determine two or more sets of strut lengths under two or more different loads. Again, each set of strut lengths yields a pose and position via calculations. The loaded pose and positions are subtracted from the unloaded pose and position to find a difference or change in each of the pose and position components as a result of the load. This strain information combined with the difference in loads yields a measure of fracture site stiffness.

In one embodiment, the method involves attaching an external fixator (such as a Taylor Spatial Frame) or similar device mechanical sensor to another (second) external fixator (such as a unilateral fixator) which is attached to the skeleton. The spanning members of the second external fixator are removed or thoroughly loosened. The offsets of the accessory or piggyback external fixator are recorded under mounting parameters and the method may be used to determine two or more sets of strut lengths under two or more different loads. Again, each set of strut lengths yields a pose and position via calculations. The loaded pose and positions are subtracted from the unloaded pose and position to find a difference or change in each of the pose and position components as a result of the load. This strain information combined with the difference in loads yields a measure of fracture site stiffness. The external fixator may be a unilateral, circular, hybrid or spatial external fixator.

In one embodiment, the strut lengths may be recorded when the external fixator is in a known load situation. The first pose and position of the external fixator may be determined when the fixator is a first known loaded situation.

In one embodiment, the external fixator may be directly loaded and subsequent translations and rotations determined to provide fracture stiffness information. However, it is usually more uncomfortable to the patient to load the bone via a ring especially with tensioned wire fixation than to apply the load to bone by standing. Also, the half pins and wires are basically a ‘spring’ in series with the fracture site's elasticity and must be accounted for when determining fracture site stiffness.

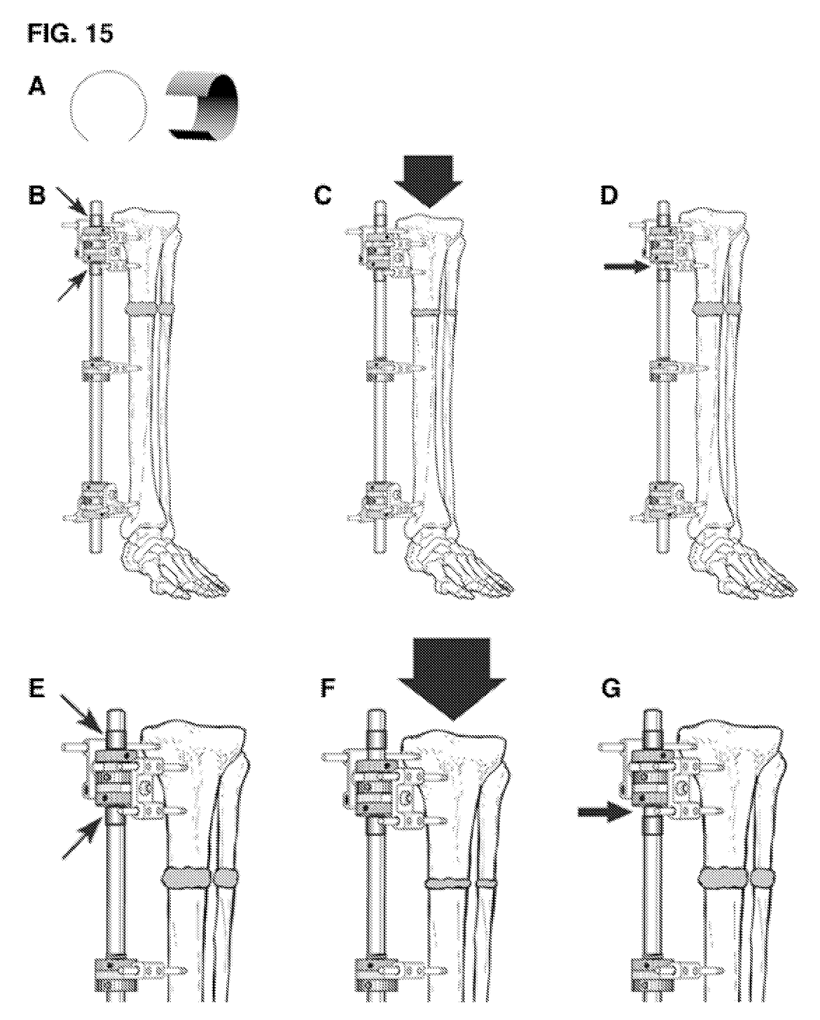

Referring now to FIG. 15, FIG. 15 represents a unilateral bar external fixator which may be a part of a more complex multifocal application also involving ring fixation. For simplicity sake, a proximal tibial fracture is illustrated with the Hex-Fix external fixator. Loosening a set screw on the proximal spool/carriage allows the proximal carriage to translate along the bar (Z-axis). Under a given load along the Z-axis, the translation of the proximal carriage with respect to the bar/other carriages is recorded. This applied load divided by displacement yields stiffness information about the fracture site. Measurements could be made with an accessory scale (ruler) or direct readings from a bar marked with indicia or by an accessory optical reader or camera. Also, an integral or accessory LVDT could be utilized to measure displacement.

FIG. 15A shows the indicator clip which would be preferably plastic or metal or other elastic material so that the clip could be applied intermediate to the ends. The clip would be tight enough about the bar that the clip would not slip under force of gravity or motion of the limb. However, the clip would slide under a small force.

Alternatively, the clip could be made of a variety of materials and be enclosed about the bar in one piece or hinged to enclose the bar. The friction prevents inadvertent sliding provided by elastic interference.

FIG. 15B shows indicator clips applied to the bar on either side of the proximal spool. The retaining set screw of the proximal spool is released to allow axial translation of the proximal spool on the bar. An axial force, either compressive or tensile is applied to the skeleton 15C. The indicator clip will translate with the spool. Assuming an elastic property of the fracture site, when the load is released the spool will return to its initial position and the space between the distal edge of the spool and the distal indicator clip is a measure of axial displacement of the proximal spool/proximal fragment 15D. FIGS. 15E, F, G are close-ups of FIGS. 15B, C, and D respectively.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the disclosed invention and equivalents thereof.

What is claimed is:

1. A method of monitoring healing and/or assessing mechanical stiffness of a bone fracture or the like, the method comprising:
a. recording a strut length of at least one variable length strut with an indicator pin, wherein said strut is on an external fixator attached to bone surrounding a bone fracture, wherein the external fixator is in a no load or minimal load situation;
b. inputting strut length from step (a), mounting parameters, and frame parameters into software to determine for a first pose and position of the fixator in the no load or minimal load situation;
c. positioning an indicator clip on at least one side of the indicator pin;
d. applying a load or torque to the bone fracture;
e. recording strut length of the at least one variable length strut in loaded situation;
f. inputting strut length from step (e), mounting parameters, and frame parameters into software to determine for a second pose and position of the fixator in a loaded situation;
g. solving for a change in pose and position by subtracting the second pose and position from the first pose and position; and
h. solving for the stiffness of the bone fracture by dividing the load applied to the bone fracture by the change in position and pose.

2. The method of claim 1, wherein the method comprises the initial step of recording initial strut length of at least one variable length strut.

3. The method of claim 1 or 2, wherein steps (a) to (h) are repeated.

4. The method of claim 1 wherein the external fixator comprises a six axis parallel manipulator type external fixator having a plurality of variable length struts.

5. The method of claim 1, wherein the external fixator is attached to a second external fixator which is attached to bone surrounding the bone fracture.

6. The method of claim 5, wherein the second external fixator is a unilateral external fixator.

7. The method of claim 5, wherein the second external fixator is a circular external fixator.

8. The method of claim 5, wherein the second external fixator is a hybrid external fixator.

9. The method of claim 5, wherein the second external fixator is a spatial external fixator.

10. The method of claim 1, wherein step (a) comprises the external fixator in a first known loaded situation and wherein step (b) determining for a first pose and position of the fixator in a first known loaded situation.

11. The method of claim 1, wherein step (c) comprises positioning an indicator clip on each side of the indicator pin.

12. The method of claim 1 wherein the indicator clip comprises a first curved portion, a second curved portion and a tab, wherein the first and second curved portions are configured to abut the body of the strut when the clip is attached to the strut, wherein the tab is contiguous to the first and second curved portions.

13. The method of claim 1 wherein the indicator clip comprises a cylindrical portion and a tab, wherein the cylindrical portion is configured to abut the body of the strut when the clip is attached to the strut, wherein the tab is contiguous to the cylindrical portion.

14. A method of monitoring healing and/or assessing mechanical stiffness of a bone fracture or the like, the method comprising:
a. recording a strut length of at least one variable length strut with an indicator pin, wherein said strut is on an external fixator attached to bone surrounding a bone fracture, wherein the external fixator is in a no load or minimal load situation;
b. inputting strut length from step (a), mounting parameters, and frame parameters into software to determine for a first pose and position of the fixator in the no load or minimal load situation;
c. applying a load or torque to the bone fracture;
d. recording strut length of the at least one variable length strut in loaded situation;
e. inputting strut length from step (d), mounting parameters, and frame parameters into software to determine for a second pose and position of the fixator in a loaded situation;
f. solving for a change in pose and position by subtracting the second pose and position from the first pose and position; and
g. solving for the stiffness of the bone fracture by dividing the load applied to the bone fracture by the change in position and pose.

15. The method of claim 14 wherein the method comprises the initial step of recording initial strut length of at least one variable length strut.

16. The method of claim 14 or 15, wherein steps (a) to (g) are repeated.

17. The method of claim 14 wherein the external fixator comprises a six axis parallel manipulator type external fixator having a plurality of variable length struts.

18. The method of claim 14, wherein the external fixator is attached to a second external fixator which is attached to bone surrounding the bone fracture.

19. The method of claim 18, wherein the second external fixator is a unilateral external fixator.

20. The method of claim 18, wherein the second external fixator is a circular external fixator.

21. The method of claim 18, wherein the second external fixator is a spatial external fixator.

22. The method of claim 18, wherein the second external fixator is a hybrid external fixator.

23. An indicator clip used to indicate the change in length of a variable length strut, wherein the strut comprises a strut body which is hollow to accept a threaded portion which telescopes into the strut body, wherein an indicator pin is attached to the threaded portion and travels along a channel cut in the body of the strut, the clip comprising:
a. a first curved portion and a second curved portion, wherein the curved portions are configured to abut the body of the strut when attached to the strut; and
b. a tab contiguous to the first and second curved portions, wherein the tab is configured to reside and travel in the channel located in the body of the variable length strut when the tab is attached to the strut.

24. The clip in claim 23, wherein the clip is permanently attached to the strut.

25. The clip in claim 23, wherein the clip is removably attached to the strut.

26. An indicator clip used to indicate the change in length of a variable length strut, wherein the strut comprises a strut body which is hollow to accept a threaded portion which telescopes into the strut body, wherein an indicator pin is attached to the threaded portion and travels along a channel cut in the body of the strut, the clip comprising:
a. a cylindrical portion configured to abut the body of the strut when attached to the strut; and
b. a tab contiguous to the cylindrical portion, wherein the tab is configured to reside and travel in the channel located in the body of the variable length strut.

27. The clip of claim 26, wherein the clip is permanently attached to the strut.

28. The clip of claim 26, wherein the clip is removably attached to the strut.

29. A method of monitoring healing and/or assessing mechanical stiffness of a bone fracture or the like, the method comprising:
a. floating an external fixator in a no load or minimal load situation, wherein the external fixator is attached to bone surrounding the bone fracture, wherein the external fixator has at least one variable length strut configured to electronically measure and transmit corresponding strut length of the at least one variable length strut into software;
b. determining for a first pose and position of the fixator in a no load or minimal load situation by inputting strut length from step (a), mounting parameters, and frame parameters into software;
c. applying a load or torque to the bone fracture;
d. electronically measure and transmit strut length of the at least one variable length strut in loaded situation;
e. determining for a second pose and position of the fixator in a loaded situation by inputting strut length from step (d), mounting parameters, and frame parameters into software;
f. solving for a change in pose and position by subtracting the second pose and position from the first pose and position; and
g. solving for the stiffness of the bone fracture by dividing the load applied to the bone fracture by the change in position and pose.

30. The method of claim 29 wherein the method comprises the initial step of electronically recording initial strut length of at least one variable length strut.

31. The method of claim 29 or 30, wherein steps (a) to (g) are repeated.

32. The method of claim 29 wherein the external fixator comprises a six axis parallel manipulator type external fixator having a plurality of variable length struts.

33. The method of claim 29, wherein the external fixator is attached to a second external fixator which is attached to bone surrounding the bone fracture.

34. The method of claim 33, wherein the second external fixator is a unilateral external fixator.

35. The method of claim 33, wherein the second external fixator is a circular external fixator.

36. The method of claim 33, wherein the second external fixator is a spatial external fixator.

37. The method of claim 33, wherein the second external fixator is a hybrid external fixator.

38. An apparatus for monitoring healing and/or assessing mechanical stiffness of a bone fracture or the like, the apparatus comprising: (a) a variable length strut, wherein the strut comprises a strut body which is hollow to accept a threaded portion which telescopes into the strut body, wherein an indicator pin is attached to the threaded portion and travels along a channel cut in the body of the strut; and (b) at least one indicator clip adjacent to at least one side of the indicator pin of the strut, wherein the clip comprises a first curved portion, a second curved portion, and a tab contiguous to the curved portions, wherein the curved portions are configured to abut the body of the strut when attached to the strut; wherein the tab is configured to reside in the channel located in the body of the strut when the tab is attached to the strut.

39. The apparatus of claim 38, wherein the variable length strut is attached to an external fixator comprising a six axis parallel manipulator type external fixator having a plurality of variable length struts.

40. The apparatus of claim 39, wherein the external fixator is attached to a second external fixator which is attached to bone surrounding the bone fracture.

41. The apparatus of claim 40, wherein the second external fixator is a unilateral external fixator.

42. The apparatus of claim 40 wherein the external fixator is a circular external fixator.

43. The apparatus of claim 40 wherein the external fixator is a hybrid external fixator.

44. The apparatus of claim 40, wherein the external fixator is a spatial fixator.

45. The apparatus of claim 38, wherein the indicator clip is permanently attached to the strut.

46. The apparatus of claim 38, wherein the indicator clip is removably attached to the strut.

46. The apparatus of claim 38, wherein an indicator clip is attached to each side of the indicator pin.

47. An indicator clip used to indicate a change in length, wherein the clip is a partial or full cylinder attachable to a bar of a unilateral fixator.

48. A method for monitoring healing and assessing stiffness of a fracture site, wherein a unilateral external fixator, having a bar, is attached to the fracture site, wherein the indicator clip of claim 47 is attached to the fixator, the method comprising:
a. loosening a screw on the proximal carriage of the external fixator;
b. applying a given load to the bar;
c. determining axial displacement by recording displacement of the indicator clip; and
d. determining stiffness of fracture site by dividing load by displacement of the indicator clip.

49. The method of claim 48 wherein the clip is removably attached to the fixator.

50. The method of claim 48 wherein the clip is permanently attached to the fixator.

51. A method of monitoring healing of a bone fracture or the like, the method comprising:
a. recording a strut length of at least one variable length strut with an indicator pin, wherein said strut is on an external fixator attached to bone surrounding a bone fracture, wherein the external fixator is in a no load or minimal load situation;
b. inputting strut length from step (a), mounting parameters, and frame parameters into software to determine for a first pose and position of the fixator in the no load or minimal load situation;
c. positioning an indicator clip on at least one side of the indicator pin;
d. applying a force to the bone fracture;
e. recording strut length of the at least one variable length strut in loaded situation;
f. inputting strut length from step (e), mounting parameters, and frame parameters into software to determine for a second pose and position of the fixator in a loaded situation; and
g. solving for a change in pose and position by subtracting the second pose and position from the first pose and position.

52. The method of claim 51, wherein the method comprises the initial step of recording initial strut length of at least one variable length strut.

53. The method of claim 51 or 52, wherein steps (a) to (g) are repeated.

54. The method of claim 51 wherein the external fixator comprises a six axis parallel manipulator type external fixator having a plurality of variable length struts.

55. The method of claim 51, wherein the external fixator is attached to a second external fixator which is attached to bone surrounding the bone fracture.

56. The method of claim 55, wherein the second external fixator is a unilateral external fixator.

57. The method of claim 55, wherein the second external fixator is a circular external fixator.

58. The method of claim 55, wherein the second external fixator is a hybrid external fixator.

59. The method of claim 55, wherein the second external fixator is a spatial external fixator.

60. The method of claim 1, wherein step (c) comprises positioning an indicator clip on each side of the indicator pin.

61. The method of claim 51 wherein the indicator clip comprises a first curved portion, a second curved portion and a tab, wherein the first and second curved portions are configured to abut the body of the strut when the clip is attached to the strut, wherein the tab is contiguous to the first and second curved portions.

62. The method of claim 51 wherein the indicator clip comprises a cylindrical portion and a tab, wherein the cylindrical portion is configured to abut the body of the strut when the clip is attached to the strut, wherein the tab is contiguous to the cylindrical portion.
